(19) United States
(12) Patent Application Publication
(10) Pub. No.: US 2025/0201354 A1
(43) Pub. Date: Jun. 19, 2025

(54) Air Panacea

(71) Applicant: Anuthep Benja-Athon, Manhasset, NY (US)

(72) Inventor: Anuthep Benja-Athon, Manhasset, NY (US)

(21) Appl. No.: 18/445,656

(22) Filed: Dec. 15, 2023

(51) Int. Cl. G16C 20/10; G06F 3/0482; G16C 20/80

(52) U.S. Cl. CPC G16C 20/10; G06F 3/0482; G16C 20/80

(57) ABSTRACT

Interactive and adaptable spectrums of pathognomonic adverse reactions in individual groupings of polypharmaceuticals associating with interactive figures, medical imagings and holograms to preventing and resolving and foretelling causal relations of said pathognomonic adverse reactions in and among billions of abiotic intelligence-networked communication devices.

This is a continuation-in-part of Ser. No. 18/445,226 entitled AIR Brain filed Jun. 7, 2023, Ser. No. 17/535,996 entitled AIR Oracle Brain filed Nov. 27, 2021, Ser. No. 15/495,164 entitled AIR Oracle filed Apr. 24, 2017, and U.S. Pat. No. 6,132,218 entitled Images for communication of medical information in computer Oct. 17, 2000.

### FIELD OF THE INVENTION

The preventions and cures for adverse reactions of pharmaceuticals

### BACKGROUND OF THE INVENTION

Prescription and over-the-counter medications-related diseases and disorders and sequelae—either unbeknownst to or not appreciated by billions of physicians and patients—commonly afflicting said physicians and patients especially those who take multiple pharmaceuticals and over long period of times. Consequently millions of patients are afflicted with said diseases and disorders and sequelae causing high morbidity and mortality and immense tangible and intangible socioeconomic losses and sufferings per annum.

The Physicians' Desk References also commonly known as PDR, at best, and its digital version online—neither provide nor able to solve said crises nor achieve the goals and objectives of the present invention.

PDR comprised about 3151 pages of 9 font words and terms size—of PDR Network, LLC at PDR.net version—is the companion and most comprehensive reference on pharmacopoeia, albeit does not achieve the goals and objectives of the present invention, all physicians who do not open PDF including said digital version unless they must; and all American physicians including this applicant use and possess the Physicians' Desk Reference.

Worse, all medical and surgical textbooks and millions of literatures, journals, publications and laboratory data sources and their digital versions have worse aforementioned deficiencies.

In light of the aforementioned, all physicians and patients are plagued by pathognomonic spectrums of pharmalogic basis of diseases and disorders of every organ with adverse sequelae, morbidity including acute and chronic diseases and disorders, mortality and socioeconomic losses and costs.

In the history of mankind, the omnipotent and omnipresent AIR Panacea comprising the interactive and adaptable spectrums for preventing and resolving pathognomonic adverse reactions and creating the actual incidences and prevalences, and extrapolating the propagations and causal relations of pathognomonic adverse reactions associating with and relating to individual medications and individual groupings of polypharmaceuticals (more than one medications in a person) in billions of people.

### SUMMARY OF THE INVENTION

Prescription and over-the-counter medications-related diseases and disorders and sequelae—either unbeknownst or not appreciated—afflicting billions of people.

AIR Panacea comprising the interactive and adaptable spectrums for preventing and resolving pathognomonic adverse reactions and creating the actual incidences and prevalences, and extrapolating the propagations and causal relations of said pathognomonic adverse reactions associating with and relating to individual medications and individual groupings of polypharmaceuticals (more than one medications in a person) in billions of people using billions of Apple iPhones, Samsung Galaxys, Google Pixels and the likes, laptops, desktops and servers.

### DESCRIPTION OF THE PREFERRED EMBODIMENTS

In Abiotic Intelligence-Rendered Panacea also known as AIR Panacea herein, a method of creating said interactive and adaptable spectrums comprising pathognomonic adverse reactions associating with and relating to individual medications and individual groupings of polypharmaceuticals relating to people's relational profile data and identifications of said abiotic intelligence-networked communication devices, whereby said interactive and adaptable spectrums associating with and relating to interactive highlighted and accentuated APS coordinates and whereby said interactive APS coordinates associating with connected constellations of connected assortments of interactive and adaptable SIVSF-silhouetted SIUNDV comprising said interactive and adaptable spectrums, whereby said interactive and adaptable spectrums preventing and resolving pathognomonic adverse reactions, creating the actual incidences and prevalences of pathognomonic adverse reactions, and extrapolating the propagations and foretells of causal relations—diseases and disorders and sequelae—of pathognomonic adverse reactions individual groupings of polypharmaceuticals relating to people's relational profile data and identifications of said abiotic intelligence-networked communication devices.

The method comprising the processes of the innovative computer programs instructing processor units to enabling physicians, scientists and professors and students to take printed or digital relational data in several Physicians' Desk References, such as, but limited to PDR 1996 edition and online, respectively, said printed or digital relational data comprising:
about 160 Pharmaceutical Product Categories such as, but not limited to, ACE inhibitors, acromegaly drug, AIDS chemotherapy agents, AIDS related complex therapeutic agents, Acne products, Adhesion barriers, Adrenal cortical steroid, alcohol abuse reduction preparations allergy relief products, etc.
said 160 Pharmaceutical Product Categories comprising pharmaceutical product category-specific 600 medications or pharmaceuticals;
said 600 medications have brand Names; and
said 600 medications have generic name of said 600 medications;

Each medication, in said 600 medications, has its pathognomonic adverse reactions in the following: Description; Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations.

Further to supra, Ser. No. 15/495,164 entitled AIR Oracle taught said physicians, scientists and professors and students to take said printed or digital relational databases to combine digital anatomical positioning system of coordinates of 2D & 3D surface, gross and microscopic human anatomy in. interactive figures, medical imagings and 2D & 3D holograms in and among viewing screens and touchscreens and ambiences for displaying interactive 2D and 3D holograms, henceforth known herein as APS coordinates. By so creating, said 600 medications in the relational databases in and among said storage memories, comprising the following algorithm:
Pharmaceutical Product Category*_m:
Pharmaceutical Generic Name*_n;
Pharmaceutical Brand Name*_n:
Frequent Pathognomonic Adverse Reaction*_p; APS coordinate*_q; adverse reaction of organ systems*;
Infrequent Pathognomonic Adverse Reaction*_r; APS coordinate*_s; adverse reaction of organ systems*;
Rare Pathognomonic Adverse Reaction*_t; APS coordinate*_u; adverse reaction of organ systems*;
said organ system* comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations; and
where * is wild, m=1-160, n=1-600, p=1-30, q=1-280, r=1-30, s=1-280, t=1-30, and u=1-280.

An example of said algorithm applicable to said 600 medications being represented by Gabapentin as follows:
Gabapentin:
Frequent PAR;111;A; hypertension;
Frequent PAR;112;A; hypertension;
Infrequent PAR;10.02;A; palpitation;
Infrequent PAR;10.02;A; tachycardia;
Infrequent PAR;111;A; hypotension;
Infrequent PAR;112;A; hypotension;
Infrequent PAR;10.02;A; angina pectoris;
Infrequent PAR;1;AS; migraine;
Infrequent PAR;10.02;A; murmur;
Infrequent PAR;20;AS; peripheral vascular disorder;
Infrequent PAR;21;AS; peripheral vascular disorder;
Rare PAR;10.02;A; arial fibrillation;
Rare PAR;10.02;A; heart failure;
Rare PAR;20;AS; thrombophlebitis;
Rare PAR;21;AS; thrombophlebitis;
Rare PAR;20;AS; deep thrombophlebitis;
Rare PAR;21;AS; deep thrombophlebitis;
Rare PAR;10.02;A; myocardial infarction;
Rare PAR;1;AS; cerebrovascular accident;
Rare PAR;10;AS; pulmonary thrombosis;
Rare PAR;10.02;A; ventricular extrasystoles;
Rare PAR;10.02;A; bradycardia;
Rare PAR;10.02;A; premature atrial contraction;
Rare PAR;10.02;A; pericardial rub;
Rare PAR;10.02;A; heart block;
Rare PAR;10;AS; pulmonary embolus;
Rare PAR;10.02;A; pericardial effusion;
Rare PAR;10.02;A; pericarditis;
Rare PAR;81;A; hyperlipidemia;
Rare PAR;81;A; hypercholesterolemia;
Column1; Column 2;Column 3; comprising:
PAR headings; anatomical positionings and anatomical views; adverse reaction of organ systems; Column 2 is the anatomical positioning system of coordinates aka APS coordinates in 2D anterior surface gross human anatomy comprising:
numerics in said 2D anterior surface gross human anatomy; there are 140 anterior anatomical positioning coordinates; and numerics in said 2D posterior surface gross human anatomy; there are 140 posterior anatomical positioning coordinates.

Ser. No. 15/495,164 entitled AIR Brain taught—an example of said 600 medications in said relational databases (APS coordinates are omitted in the next paragraph as they are in another albeit related relational data in and among said storage memories) being represented by Gabapentin as follows:
Generic Name—Gabapentin;
Product Name—Neurotin:
Cardiovascular System:
Pathognomonic Adverse Reactions:
Frequent Pathognomonic Adverse Reactions: hypertension;
Infrequent Pathognomonic Adverse Reactions: palpitation, tachycardia, hypotension, angina pectoris, migraine, murmur; peripheralvascular disorder;
Rare Pathognomonic Adverse Reactions: arial fibrillation, heart failure, thrombophlebitis, deep thrombophlebitis, myocardial infarction, cerebrovascular accident, pulmonary thrombosis, ventricular extrasystoles, bradycardia, premature atrial contraction, pericardial rub, heart block, pulmonary embolus, hyperlipidemia, hypercholesterolemia, pericardial effusion, pericarditis. Note: Most of said Frequent Pathognomonic Adverse Reactions, Infrequent Pathognomonic Adverse Reactions and Rare Pathognomonic Adverse Reaction are absent in PDRs. Thus, the significance of innovative “Actual . . . ” below and in the Claims herein can be understood.

In AIR Panacea, a method of creating said interactive and adaptable spectrums comprising pathognomonic adverse reactions associating with and relating to individual medications and individual groupings of polypharmaceuticals relating to people's relational profile data and identifications of said abiotic intelligence-networked communication devices, whereby said interactive and adaptable spectrums associating with and relating to interactive highlighted and accentuated APS coordinates and whereby said interactive APS coordinates associating with connected constellations of connected assortments of interactive and adaptable SIVSF-silhouetted SIUNDV comprising said interactive and adaptable spectrums,
whereby said interactive and adaptable spectrums preventing and resolving pathognomonic adverse reactions, creating the actual incidences and prevalences of pathognomonic adverse reactions, and extrapolating the propagations and foretells of causal relations—diseases and disorders and sequelae—of pathognomonic adverse reactions individual groupings of polypharmaceuticals relating to people's relational profile data and identifications of said abiotic intelligence-networked communication devices.

The method comprising the processes of creating and storing the relational databases whereby physicians', scientists', professors' and students' abiotic intelligence-networked communication devices keyboarding and merging printed or digital relational data in Physicians' Desk References, readily available to and possessed by millions of them, with APS coordinates, in said relational databases, comprising the following algorithm:
Pharmaceutical Product Categories:
Pharmaceutical Generic Names;
Pharmaceutical Brand Names:
Pathognomonic Adverse Reactions; APS coordinates; adverse reaction of organ systems;
said organ systems comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations.

The method comprising the processes of creating and storing the relational databases whereby interactive and adaptable spectrums of pharmacologic basis of pathognomonic adverse reactions associating with and relating to individual medications associating with and relating to interactive highlighted and accentuated APS coordinates, comprising:
a plurality of interactive digital scrolls effecting said processor units creating and adapting said interactive and adaptable spectrums comprising:
interactive digital scrolls displaying list of organ systems;
interactive digital scrolls displaying list of organ systems specific-pathognomonic adverse reactions; and
said interactive digital scrolls-created relational data comprising organ systems relating to organ systems specific-pathognomonic adverse reactions:
Pharmaceutical Product Category:
Pharmaceutical Generic Name;
Pharmaceutical Brand Name:
Pathognomonic Adverse Reactions; APS coordinates; adverse reaction of organ systems;
people's relational profile data and identifications of abiotic intelligence-networked communication devices;

A case in point, a user will use said: interactive digital scrolls displaying list of organ systems to select Cardiovascular System;
interactive digital scrolls displaying list of organ systems specific-pathognomonic adverse reactions to select angina pectoris; and
said interactive digital scrolls-created relational data, comprising said Cardiovascular System and angina pectoris, effecting said processor units creating and adapting said interactive and adaptable spectrums comprising said pathognomonic adverse reactions associating with and relating to individual medications associating with said Cardiovascular System and angina pectoris, and all Pharmaceutical Product Category, Pharmaceutical Generic Name, Pharmaceutical Brand Name, associating with said Cardiovascular System and angina pectoris, with and relating to interactive highlighted and accentuated APS coordinates according to said algorithm.
said interactive highlighted and accentuated APS coordinate associating with connected constellations of connected assortments of interactive and adaptable SIVSF-silhouetted SIUNDV comprising said interactive and adaptable spectrums.

Ser. No. 15/495,164 entitled AIR Oracle taught:
said interactive and adaptable SIVSF comprising individual pharmaceutical wherein each medication comprising the following algorithm:
Pharmaceutical Product Category:
Pharmaceutical Generic Name;
Pharmaceutical Brand Name:
Actual Frequent Pathognomonic Adverse Reactions; adverse reaction of organ systems;
Actual Infrequent Pathognomonic Adverse Reactions; adverse reaction of organ systems;
Actual Rare Pathognomonic Adverse Reactions; adverse reaction of organ systems;
said interactive and adaptable silhouetted SIUNDV, comprising the following algorithm, comprising said 600 pharmaceuticals:
Pharmaceutical Product Category*_m:
Pharmaceutical Generic Name*_n;
Pharmaceutical Brand Name*_n:
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
wherein said interactive and adaptable SIVSF silhouetting said interactive and adaptable silhouetted SIUNDV in touchscreens and viewing screens in and among billions of abiotic intelligence-networked communication devices as taught by Ser. No. 15/495,164 entitled AIR Oracle.

The method comprising said processes of interactive and adaptable spectrums creating the actual incidences and prevalences of pathognomonic adverse reactions associating with and relating to interactive highlighted and accentuated APS coordinates comprising:
said interactive and adaptable spectrums of pharmacologic basis of pathognomonic adverse reactions associating with and relating to individual medications associating with and relating to interactive highlighted and accentuated APS coordinates incidences and prevalences of said interactive digital scrolls-created relational data, relating to people's relational profile data and identifications of abiotic intelligence-networked communication devices, in and shared among said storage memories;
said interactive digital scrolls-created relational data effecting said processor units creating compilations of said incidences and prevalences of the same pathognomonic adverse reactions associating with and relating to individual same APS coordinates, wherein visually highlighted and accentuated individual same pathognomonic adverse reactions associating with and relating to individual same interactive highlighted and accentuated APS coordinates;
commercial software including commercial statistical and actuarial software conferring statistical confidences of said compilations of actual incidences and prevalences of said Actual Frequent Pathognomonic Adverse Reaction*_p, Actual Infrequent Pathognomonic Adverse Reaction*_r and Actual Rare Pathognomonic Adverse Reaction*_t.

The method comprising said processes of interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates, comprising:
said interactive digital scrolls effecting said processor units creating said interactive and adaptable spectrums creating the actual incidences and prevalences and propagations of causal relations—diseases and disorders and sequelae—of said pathognomonic adverse reactions, comprising:
interactive digital scrolls displaying list of interactive pharmaceutical product categories;
interactive digital scrolls displaying list of interactive digital scrolls pharmaceutical brand names; and
interactive digital scrolls displaying list of interactive digital scrolls pharmaceutical generic names;
select buttons creating and adapting selections of said relational data in said plurality of interactive digital scrolls;
“x” buttons deleting thence adapting said selections;
submit buttons submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical brand names relating to pharmaceutical generic names in said relational databases;
said interactive digital scrolls-created relational data effecting said processor units getting relational data relating to said interactive digital scrolls-created relational data in said relational databases;
said computer programs instructing processor units:
scanning for same pathognomonic adverse reactions in relational data relating to said brand names relating to a person's relational profile data and identification of abiotic intelligence-networked communication devices;
scanning for same pathognomonic adverse reactions in relational data relating to said generic names relating to a person's relational profile data and identification of abiotic intelligence-networked communication devices;
creating said interactive and adaptable spectrums associating with and relating to interactive highlighted and accentuated APS coordinates wherein same pathognomonic adverse reactions associating with and relating to interactive highlighted and accentuated APS coordinates in and among in figures, medical imagings and 2D & 3D holograms in and among viewing screens and touchscreens and ambiences displaying interactive 2D and 3D holograms;
same pathognomonic adverse reactions associating with and relating to interactive highlighted and accentuated APS coordinates in individual groupings of plurality of medications comprising Pharmaceutical Product Categories, Pharmaceutical brand names and Pharmaceutical generic names relating to people's relational profile data and identifications of said abiotic intelligence-networked communication devices;
pharmaceutical Product Categories, Pharmaceutical brand names and Pharmaceutical generic names and medications-specific pathognomonic adverse reactions associating with and relating to interactive highlighted and accentuated APS coordinates, in individual polypharmaceuticals relating to individual persons and identifications of said abiotic intelligence-networked communication devices; and
said people's relational profile data; and
said identifications of said abiotic intelligence-networked communication devices.

A case in point, in person being prescribed and ingesting 4 medications or individual grouping of polypharmaceuticals comprising Gabapentin, Amphetamine, Ibuprofen and Isosorbide—comprising same pathognomonic adverse reactions which are hypertension, palpitation, tachycardia relating to Cardiovascular System—by his/her physicians, using only 2 representative Cardiovascular System and Digestive System of said 4 medications, representing billions of people using billions of smartphones, according to printed relational data in PDR 1996, said relational databases are represented:
Generic Name—Gabapentin;
Product Name—Neurotin:
Cardiovascular System:
Pathognomonic Adverse Reactions:
Frequent Pathognomonic Adverse Reactions: hypertension;
Infrequent Pathognomonic Adverse Reactions: palpitation, tachycardia, hypotension, angina pectoris, migraine, murmur; peripheralvascular disorder;
Rare Pathognomonic Adverse Reactions: arial fibrillation, heart failure, thrombophlebitis, deep thrombophlebitis, myocardial infarction, cerebrovascular accident, pulmonary thrombosis, ventricular extrasystoles, bradycardia, premature atrial contraction, pericardial rub, heart block, pulmonary embolus, hyperlipidemia, hypercholesterolemia, pericardial effusion, pericarditis.
Generic Name—Gabapentin;
Product Name—Neurotin:
Digestive System:
Pathognomonic Adverse Reactions:
Frequent Pathognomonic Adverse Reactions: anorexia, flatulence, gingivitis; Infrequent Pathognomonic Adverse Reactions: glossitis, gum hemorrhage, thirst, stomatitis, increased salivation, gastroenteritis; hemorrhoids, bloody stools, fecal incontinence, hepatomegaly;
Rare Pathognomonic Adverse Reactions: dysphagia, eructation, pancreatitis, peptic ulcer, colitis, mouth blisters, tooth discolor, perleche, salivary gland enlarged, lip hemorrhage, esophagitis, hiatal hernia, hematemesis, proctitis, irritable bowel syndrome, rectal hemorrhage, esophageal spasm.
Generic Name—Biphetamine;
Product Name—Amphetamine:
Cardiovascular System:
Pathognomonic Adverse Reactions: Cardiovascular System: palpitation, hypertension, tachycardia, cardiomyopathy.
Generic Name—Biphetamine;
Product Name—Amphetamine:
Digestive System:
Pathognomonic Adverse Reactions: mouth dryness, unpleasant taste, diarrhea, constipation, other gastrointestinal disturbances.
Generic Name—Ibuprofen;
Product Name—Motrin:
Cardiovascular System:
Pathognomonic Adverse Reactions:
Frequent Pathognomonic Adverse Reactions: edema, fluid retention;
Infrequent Pathognomonic Adverse Reactions: palpitation, hypertension, congestive heart failure; tachycardia, sinus tachycardia, sinus bradycardia, arrhythmia.
Generic Name—Ibuprofen;
Product Name—Motrin:
Digestive System:
Pathognomonic Adverse Reactions:
Frequent Pathognomonic Adverse Reactions: nausea, epigastric pain, heartburn, diarrhea, abdominal distress, vomiting, indigestion, constipation, abdominal pain, abdominal cramps, GI tract fullness, bloating, flatulence;
Infrequent Pathognomonic Adverse Reactions: gastric ulcer, duodenal ulcer, gastric bleeding, duodenal bleeding, gastrointestinal hemorrhage, melena, gastritis, hepatitis, jaundice, abnormal liver function test, pancreatitis.
Generic Name—Isosorbide:
Product Name—Imdur:
Cardiovascular System:
Pathognomonic Adverse Reactions: hypertension; palpitation, tachycardia, hypotension, bradycardia; extrasystole, atrial fibrillation, ventricular tachycardia, arrhythmia, bundle branch block, cardiac failure.
Generic Name—Isosorbide:
Product Name—Imdur:
Digestive System:
Pathognomonic Adverse Reactions: abdominal pain, constipation, diarrhea, dyspepsia, flatulence, gastric ulcer, gastritis, glossitis, hemorrhagic gastric ulcer, hemorrhoids, loose stools; melena, nausea, vomiting.

The method comprising said processes of interactive and adaptable spectrums preventing and resolving pathognomonic adverse reactions comprising:
said interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates.

The method comprising said processes of connected constellations of connected assortments of interactive and adaptable SIVSF-silhouetted SIUNDV comprising:
said interactive and adaptable spectrums associating with and relating to interactive highlighted and accentuated APS coordinates
interactive highlighted and accentuated APS coordinates associating with said connected constellations of connected assortments of interactive and adaptable SIVSF-silhouetted SIUNDV comprising said interactive and adaptable spectrums, comprising:
said interactive and adaptable SIVSF comprising individual groupings of polypharmaceuticals wherein each medication comprising the following algorithm:
Pharmaceutical Product Category:
Pharmaceutical Generic Name;
Pharmaceutical Brand Name:
Actual Frequent Pathognomonic Adverse Reactions; adverse reaction of organ systems;
Actual Infrequent Pathognomonic Adverse Reactions; adverse reaction of organ systems;
Actual Rare Pathognomonic Adverse Reactions; adverse reaction of organ systems;
said interactive and adaptable silhouetted SIUNDV, comprising said 600 pharmaceuticals, comprising the following algorithm:
Pharmaceutical Product Category*_m:
Pharmaceutical Generic Name*_n;
Pharmaceutical Brand Name*_n:
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
whereby said interactive and adaptable SIVSF silhouetting said interactive and adaptable silhouetted SIUNDV.

The method comprising said processes of interactive and adaptable spectrums extrapolating the propagations and foretells of causal relations—diseases and disorders and sequelae—of pathognomonic adverse reactions in individual groupings of polypharmaceuticals associating with and relating to interactive highlighted and accentuated APS coordinates comprising:
said interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates, comprising the following algorithm:
Pharmaceutical Product Category*_m;
Pharmaceutical Generic Name*_n;
Pharmaceutical Brand Name*_n;
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
interactive digital scrolls displaying list of interactive pharmaceutical product categories;
interactive digital scrolls displaying list of interactive digital scrolls pharmaceutical brand names; and
interactive digital scrolls displaying list of interactive digital scrolls pharmaceutical generic names;
select buttons creating and adapting selections of said relational data among said plurality of interactive digital scrolls;
“x” buttons deleting thence adapting said selections;
submit buttons submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical generic names in said relational databases;
said interactive digital scrolls-created relational data effecting said processor units creating compilations of said incidences and prevalences of said actual, infrequent and rare pathognomonic adverse reactions associating with and relating to each and every pharmaceutical in individual grouping of polypharmaceuticals;
compilations of said incidences and prevalences of propagations and foretells of causal relations—diseases and disorders and sequelae—in individual groupings of polypharmaceuticals in said relational databases;
commercial software including commercial statistical and actuarial software conferring statistical confidences of said extrapolations of the propagations and foretells of causal relations.

1. A system of abiotic intelligence-networked communication devices for creating interactive and adaptable spectrums of pharmacologic basis of pathognomonic adverse reactions associating with and relating to individual medications and individual groupings of polypharmaceuticals relating to people's relational profile data and identifications of said abiotic intelligence-networked communication devices wherein said interactive and adaptable spectrums for associating with and relating to interactive highlighted and accentuated APS coordinates, and said interactive APS coordinates for associating with connected constellations of connected assortments of interactive and adaptable SIVSF-silhouetted SIUNDV comprising said interactive and adaptable spectrums for preventing and resolving pathognomonic adverse reactions, said interactive and adaptable spectrums for creating the actual incidences and prevalences of pathognomonic adverse reactions, and said interactive and adaptable spectrums for extrapolating the propagations and foretells of causal relations of pathognomonic adverse reactions in said individual groupings of polypharmaceuticals, henceforth known as AIR Panacea comprising:
the abiotic intelligence-networked communication devices comprising networked servers, billions of mobile communication devices and desktops comprising said touchscreens and viewing screens and processor units attaching to storage memories;
the abiotic intelligence comprising the processor units attaching to said storage memories for storing the innovative computer programs for instructing processor units for creating said interactive and adaptable spectrums;
the relational databases;
a plurality of interactive digital scrolls for effecting said processor units for creating and adapting said interactive and adaptable spectrums;
interactive and adaptable spectrums of pharmacologic basis of pathognomonic adverse reactions associating with and relating to individual medications for associating with and relating to interactive highlighted and accentuated APS coordinates;
interactive and adaptable spectrums for creating the actual incidences and prevalences of pathognomonic adverse reactions for associating with and relating to interactive highlighted and accentuated APS coordinates;
interactive and adaptable spectrums of pharmacologic basis of pathognomonic adverse reactions associating with and relating to individual groupings of polypharmaceuticals, wherein said interactive and adaptable spectrums for associating with and relating to interactive highlighted and accentuated APS coordinates;
interactive APS coordinates for associating with connected constellations of connected assortments of interactive and adaptable SIVSF-silhouetted SIUNDV;
interactive and adaptable spectrums for preventing and resolving pathognomonic adverse reactions;
a plurality of interactive digital scrolls for effecting said processor units for creating said interactive and adaptable spectrums for creating the actual incidences and prevalences of pathognomonic adverse reactions, and said interactive and adaptable spectrums for extrapolating the propagations and foretells of causal relations of pathognomonic adverse reactions;
interactive and adaptable spectrums for extrapolating the propagations and foretells of causal relations of pathognomonic adverse reactions in individual groupings of polypharmaceuticals for associating with and relating to interactive highlighted and accentuated APS coordinates; and
commercial software including commercial statistical and actuarial software for conferring statistical confidences of said actual incidences and prevalences, and said propagations and causal relations of said pathognomonic adverse reactions.

2. The AIR Panacea according to claim 1 wherein said abiotic intelligence comprising the processor units attaching to said storage memories for storing the innovative computer programs for instructing processor units for:
creating said relational databases;
a plurality of interactive digital scrolls for effecting said processor units for creating and adapting. said interactive and adaptable spectrums;
creating said interactive and adaptable spectrums of pharmacologic basis of pathognomonic adverse reactions associating with and relating to individual medications for associating with and relating to interactive highlighted and accentuated APS coordinates;
creating said interactive and adaptable spectrums for creating the actual incidences and prevalences of pathognomonic adverse reactions for associating with and relating to interactive highlighted and accentuated APS coordinates;
creating said interactive and adaptable spectrums of pharmacologic basis of pathognomonic adverse reactions associating with and relating to individual groupings of polypharmaceuticals, wherein said interactive and adaptable spectrums for associating with and relating to interactive highlighted and accentuated APS coordinates;
creating said interactive APS coordinates for associating with connected constellations of connected assortments of interactive and adaptable SIVSF-silhouetted SIUNDV;
creating said interactive and adaptable spectrums for preventing and resolving pathognomonic adverse reactions;
creating said interactive and adaptable spectrums for extrapolating the propagations and foretells of causal relations of pathognomonic adverse reactions in individual groupings of polypharmaceuticals for associating with and relating to interactive highlighted and accentuated APS coordinates;
using commercial software including commercial statistical and actuarial software for conferring statistical confidences of said actual incidences and prevalences, and said propagations and causal relations of said pathognomonic adverse reactions.

3. The AIR Panacea according to claim 1 wherein said relational databases comprising the following algorithm:
Pharmaceutical Product Category*_m:
Pharmaceutical Generic Name*_n;
Pharmaceutical Brand Name*_n:
Pathognomonic Adverse Reaction*_p; APS coordinate*_q; adverse reaction of organ systems*;
said organ system* comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations; and
said pathognomonic adverse reactions relating to said APS coordinates; and
people's relational profile data and identifications of abiotic intelligence-networked communication devices.

4. The AIR Panacea according to claim 1 wherein said plurality of interactive digital scrolls for effecting said processor units for creating and adapting said interactive and adaptable spectrums, said plurality of interactive digital scrolls comprising:
interactive digital scrolls for displaying list of interactive pharmaceutical product categories;
interactive digital scrolls for displaying list of pharmaceutical brand names of medications;
interactive digital scrolls for displaying list of pharmaceutical generic names of said medications;
interactive digital scrolls for displaying list of organ systems; and
interactive digital scrolls for displaying list of organ systems specific-pathognomonic adverse reactions;
select buttons for creating and adapting selections of said relational data associating with said plurality of interactive digital scrolls;
“x” buttons for deleting thence adapting said selections;
submit buttons for submitting said selections into said relational databases in and shared among said storage memories;
said interactive digital scrolls-created relational data comprising organ systems relating to organ systems specific-pathognomonic adverse reactions;
categories relating to pharmaceutical brand names in said relational databases;
said interactive digital scrolls-created relational data comprising pharmaceutical product categories relating to pharmaceutical generic names in said relational databases; and
said organ systems comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations.

5. The AIR Panacea according to claim 1 wherein said interactive and adaptable spectrums of pharmacologic basis of pathognomonic adverse reactions associating with and relating to individual medications for associating with and relating to interactive highlighted and accentuated APS coordinates comprising:
said plurality of interactive digital scrolls for effecting said processor units for creating and adapting said interactive and adaptable spectrums comprising:
interactive digital scrolls for displaying list of organ systems;
interactive digital scrolls for displaying list of organ systems specific-pathognomonic adverse reactions; and
said interactive digital scrolls-created relational data comprising organ systems relating to organ systems specific-pathognomonic adverse reactions, comprising the following algorithm:
Pharmaceutical Product Category:
Pharmaceutical Generic Name;
Pharmaceutical Brand Name:
Pathognomonic Adverse Reactions; APS coordinates; adverse reaction of organ systems;
people's relational profile data and identifications of abiotic intelligence-networked communication devices;
said interactive digital scrolls-created relational data for effecting said processor units for creating and adapting said interactive and adaptable spectrums comprising said pathognomonic adverse reactions associating with and relating to individual medications for associating with and relating to interactive highlighted and accentuated APS coordinates; and
said interactive APS coordinates for associating with connected constellations of connected assortments of interactive and adaptable SIVSF-silhouetted SIUNDV comprising said interactive and adaptable spectrums.

6. The interactive and adaptable SIVSF-silhouetted SIUNDV according to claim 5 comprising:
said interactive and adaptable SIVSF comprising the following algorithm:
Pharmaceutical Product Category:
Pharmaceutical Generic Name;
Pharmaceutical Brand Name:
Pathognomonic Adverse Reactions; APS coordinates; adverse reaction of organ systems;
said interactive and adaptable silhouetted SIUNDV comprising the following algorithm:
Pharmaceutical Product Category*_m:
Pharmaceutical Generic Name*_n;
Pharmaceutical Brand Name*_n:
Pathognomonic Adverse Reaction*_p; APS coordinate*_q; adverse reaction of organ systems*;
wherein said interactive and adaptable SIVSF for silhouetting said interactive and adaptable silhouetted SIUNDV.

7. The AIR Panacea according to claim 1 wherein said interactive and adaptable spectrums for creating the actual incidences and prevalences of pathognomonic adverse reactions for associating with and relating to interactive highlighted and accentuated APS coordinates comprising:
said interactive and adaptable spectrums of pharmacologic basis of pathognomonic adverse reactions associating with and relating to individual medications for associating with and relating to interactive highlighted and accentuated APS coordinates;
incidences and prevalences of said interactive digital scrolls-created relational data, relating to people's relational profile data and identifications of abiotic intelligence-networked communication devices, in and shared among said storage memories;
said interactive digital scrolls-created relational data for effecting said processor units for creating compilations of said incidences and prevalences of the same pathognomonic adverse reactions for associating with and relating to individual same APS coordinates,
wherein visually highlighted and accentuated individual same pathognomonic adverse reactions for associating with and relating to individual same interactive highlighted and accentuated APS coordinates, comprising the following algorithm:
Pharmaceutical Product Category*_m;
Pharmaceutical Generic Name*_n;
Pharmaceutical Brand Name*_n;
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
people's relational profile data and said identifications of said abiotic intelligence-networked communication devices;
commercial software including commercial statistical and actuarial software for conferring statistical confidences of said compilations of actual incidences and prevalences of said Actual Frequent Pathognomonic Adverse Reaction*_p, Actual Infrequent Pathognomonic Adverse Reaction*_r and Actual Rare Pathognomonic Adverse Reaction*_t.

8. The AIR Panacea according to claim 1 wherein said interactive and adaptable spectrums of pharmacologic basis of pathognomonic adverse reactions associating with and relating to individual groupings of polypharmaceuticals, wherein said interactive and adaptable spectrums for associating with and relating to interactive highlighted and accentuated APS coordinates, comprising:
said relational databases comprising the following algorithm:
Pharmaceutical Product Category*_m:
Pharmaceutical Generic Name*_n;
Pharmaceutical Brand Name*_n:
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
said interactive digital scrolls for effecting said processor units for creating said interactive and adaptable spectrums for creating the actual incidences and prevalences and propagations of causal relations of said pathognomonic adverse reactions, comprising:
interactive digital scrolls for displaying list of interactive pharmaceutical product categories;
interactive digital scrolls for displaying list of interactive digital scrolls pharmaceutical brand names and interactive digital scrolls for displaying list of interactive digital scrolls pharmaceutical generic names;
select buttons for creating and adapting selections of said relational data in said plurality of interactive digital scrolls;
“x” buttons for deleting thence adapting said selections;
submit buttons for submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical brand names relating to pharmaceutical generic names in said relational databases;
said interactive digital scrolls-created relational data for effecting said processor units for getting relational data relating to said interactive digital scrolls-created relational data in said relational databases;
said computer programs for instructing processor units for:
scanning for same pathognomonic adverse reactions in relational data relating to said brand names relating to a person's relational profile data and identification of abiotic intelligence-networked communication devices;
scanning for same pathognomonic adverse reactions in relational data relating to said generic names relating to a person's relational profile data and identification of abiotic intelligence-networked communication devices;
creating said interactive and adaptable spectrums for associating with and relating to interactive highlighted and accentuated APS coordinates wherein same pathognomonic adverse reactions for associating with and relating to interactive highlighted and accentuated APS coordinates in and among in figures, medical imagings and 2D & 3D holograms in and among viewing screens and touchscreens and ambiences for displaying interactive 2D and 3D holograms;
same pathognomonic adverse reactions for associating with and relating to interactive highlighted and accentuated. APS coordinates in individual groupings of plurality of medications comprising Pharmaceutical Product Categories, Pharmaceutical brand names and Pharmaceutical generic names relating to people's relational profile data and identifications of said abiotic intelligence-networked communication devices;
Pharmaceutical Product Categories, Pharmaceutical brand names and Pharmaceutical generic names and medications-specific pathognomonic adverse reactions for associating with and relating to interactive highlighted and accentuated APS coordinates, in individual polypharmaceuticals relating to individual persons and identifications of said abiotic intelligence-networked communication devices; and
said people's relational profile data; and
said identifications of said abiotic intelligence-networked communication devices.

9. The AIR Pay according to claim 1 wherein said interactive and adaptable spectrums for preventing and resolving pathognomonic adverse reactions comprising:
said interactive and adaptable spectrums of pharmacologic basis of pathognomonic adverse reactions associating with and relating to individual groupings of polypharmaceuticals, wherein said interactive and adaptable spectrums for associating with and relating to interactive highlighted and accentuated APS coordinates.

10. The AIR Panacea according to claim 1 wherein said connected constellations of connected assortments of interactive and adaptable SIVSF-silhouetted SIUNDV comprising:
said interactive and adaptable spectrums for associating with and relating to interactive highlighted and accentuated APS coordinates;
interactive highlighted and accentuated APS coordinates for associating with said connected constellations of connected assortments of interactive and adaptable SIVSF-silhouetted SIUNDV comprising said interactive and adaptable spectrums, comprising:
said interactive and adaptable SIVSF comprising individual groupings of polypharmaceuticals wherein each medication comprising the following algorithm:
Pharmaceutical Product Category:
Pharmaceutical Generic Name;
Pharmaceutical Brand Name:
Actual Frequent Pathognomonic Adverse Reactions; adverse reaction of organ systems;
Actual Infrequent Pathognomonic Adverse Reactions; adverse reaction of organ systems;
Actual Rare Pathognomonic Adverse Reactions; adverse reaction of organ systems;
said interactive and adaptable silhouetted SIUNDV comprising the following algorithm:
Pharmaceutical Product Category*_m:
Pharmaceutical Generic Name*_n;
Pharmaceutical Brand Name*_n:
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
wherein said interactive and adaptable SIVSF for silhouetting said interactive and adaptable silhouetted SIUNDV; and
said organ systems comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations.

11. The AIR Panacea according to claim 1 wherein said interactive and adaptable spectrums for extrapolating the propagations and foretells of causal relations of pathognomonic adverse reactions in individual groupings of polypharmaceuticals for associating with and relating to interactive highlighted and accentuated APS coordinates comprising:
said interactive and adaptable spectrums of pharmacologic basis of pathognomonic adverse reactions associating with and relating to individual groupings of polypharmaceuticals, wherein said interactive and adaptable spectrums for associating with and relating to interactive highlighted and accentuated APS coordinates, comprising the following algorithm:
Pharmaceutical Product Category*_m;
Pharmaceutical Generic Name*_n;
Pharmaceutical Brand Name*_n;
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
interactive digital scrolls for displaying list of interactive pharmaceutical product categories;
interactive digital scrolls for displaying list of interactive digital scrolls pharmaceutical brand names; and
interactive digital scrolls for displaying list of interactive digital scrolls pharmaceutical generic names;
select buttons for creating and adapting selections of said relational data among said plurality of interactive digital scrolls;
“x” buttons for deleting thence adapting said selections;
submit buttons for submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical generic names in said relational databases;
said interactive digital scrolls-created relational data for effecting said processor units for creating compilations of said incidences and prevalences of said actual, infrequent and rare pathognomonic adverse reactions for associating with and relating to each and every pharmaceutical in individual grouping of polypharmaceuticals;
compilations of said incidences and prevalences of propagations and foretells of causal relations in individual groupings of polypharmaceuticals in said relational databases;
commercial software including commercial statistical and actuarial software for conferring statistical confidences of said extrapolations of the propagations and foretells of causal relations.

12. In AIR Panacea, a method of creating said interactive and adaptable spectrums comprising pathognomonic adverse reactions associating with and relating to individual medications and individual groupings of polypharmaceuticals relating to people's relational profile data and identifications of said abiotic intelligence-networked communication devices,
whereby said interactive and adaptable spectrums associating with and relating to interactive highlighted and accentuated APS coordinates and whereby said interactive APS coordinates associating with connected constellations of connected assortments. of interactive and adaptable SIVSF-silhouetted SIUNDV comprising said interactive and adaptable spectrums,
whereby said interactive and adaptable spectrums preventing and resolving pathognomonic adverse reactions, creating the actual incidences and prevalences of pathognomonic adverse reactions, and extrapolating the propagations and foretells of causal relations of pathognomonic adverse reactions individual groupings of polypharmaceuticals relating to people's relational profile data and identifications of said abiotic intelligence-networked communication devices, said method comprising the processes of the innovative computer programs instructing processor units:
creating and storing the relational databases in and shared among storage memories;
creating plurality of interactive digital scrolls effecting said processor units creating and adapting said interactive and adaptable spectrums;
creating said people's relational profile data and identifications of abiotic intelligence-networked communication devices networking abiotic intelligence-networked communication devices;
creating a plurality of interactive digital scrolls effecting said processor units creating and adapting said interactive and adaptable spectrums;
creating said interactive and adaptable spectrums associating with and relating to interactive highlighted and accentuated APS coordinates;
creating said interactive APS coordinates associating with connected constellations of connected assortments of interactive and adaptable SIVSF-silhouetted SIUNDV comprising said interactive and adaptable spectrums;
a plurality of interactive digital scrolls effecting said processor units creating said interactive and adaptable spectrums preventing and resolving pathognomonic adverse reactions
creating a plurality of interactive digital scrolls effecting said processor units creating said interactive and adaptable spectrums preventing and resolving pathognomonic adverse reactions;
creating a plurality of interactive digital scrolls effecting said processor units creating said interactive and adaptable spectrums creating the actual incidences and prevalences of pathognomonic adverse reactions, and said interactive and adaptable spectrums extrapolating the propagations and foretells of causal relations of pathognomonic adverse reactions;
using commercial software including commercial statistical and actuarial software conferring statistical confidences of said actual incidences and prevalences, and extrapolations of propagations and causal relations of said pathognomonic adverse reactions.

13. The method according to claim 12 whereby said processes of creating and storing the relational databases comprising:
physicians', scientists', professors' and students' abiotic intelligence-networked communication devices keyboarding and merging printed or digital relational data in Physicians' Desk References with APS coordinates, in said relational databases, comprising the following algorithm:
Pharmaceutical Product Categories:
Pharmaceutical Generic Names;
Pharmaceutical Brand Names:
Pathognomonic Adverse Reactions; APS coordinates; adverse reaction of organ systems;
said organ systems comprising: Body As A Whole; Cardiovascular System; Digestive System; Endocrine System; Hematologic and Lymphatic Systems; Musculoskeletal System; Nervous System; Respiratory System; Skin and Appendages; Dermatological; Urogenital System; Special Senses; Drug Interactions; and Laboratory Deviations.

14. The method according to claim 12 whereby said processes of interactive and adaptable spectrums of pharmacologic basis of pathognomonic adverse reactions associating with and relating to individual medications associating with and relating to interactive highlighted and accentuated APS coordinates, comprising:
a plurality of interactive digital scrolls effecting said processor units creating and adapting said interactive and adaptable spectrums comprising:
interactive digital scrolls displaying list of organ systems;
interactive digital scrolls displaying list of organ systems specific-pathognomonic adverse. reactions; and
said interactive digital scrolls-created relational data comprising organ systems relating to organ systems specific-pathognomonic adverse reactions:
Pharmaceutical Product Category:
Pharmaceutical Generic Name;
Pharmaceutical Brand Name:
Pathognomonic Adverse Reactions; APS coordinates; adverse reaction of organ systems; people's relational profile data and identifications of abiotic intelligence-networked communication devices;
said interactive digital scrolls-created relational data effecting said processor units creating and adapting said interactive and adaptable spectrums comprising said pathognomonic adverse reactions associating with and relating to individual medications associating with and relating to interactive highlighted and accentuated APS coordinates; and
said interactive APS coordinates associating with connected constellations of connected assortments of interactive and adaptable SIVSF-silhouetted SIUNDV comprising said interactive and adaptable spectrums.

15. The method according to claim 12 whereby said processes of interactive and adaptable spectrums creating the actual incidences and prevalences of pathognomonic adverse reactions associating with and relating to interactive highlighted and accentuated APS coordinates comprising:
said interactive and adaptable spectrums of pharmacologic basis of pathognomonic adverse reactions associating with and relating to individual medications associating with and relating to interactive highlighted and accentuated APS coordinates;
incidences and prevalences of said interactive digital scrolls-created relational data, relating to people's relational profile data and identifications of abiotic intelligence-networked communication devices, in and shared among said storage memories;
said interactive digital scrolls-created relational data effecting said processor units creating compilations of said incidences and prevalences of the same pathognomonic adverse reactions associating with and relating to individual same APS coordinates, wherein visually highlighted and accentuated individual same pathognomonic adverse reactions associating with and relating to individual same interactive highlighted and accentuated APS coordinates;
commercial software including commercial statistical and actuarial software conferring statistical confidences of said compilations of actual incidences and prevalences of said Actual Frequent Pathognomonic Adverse Reaction*_p, Actual Infrequent Pathognomonic Adverse Reaction*_r and Actual Rare Pathognomonic Adverse Reaction*_t.

16. The method according to claim 12 whereby said processes of interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates, comprising:
said interactive digital scrolls effecting said processor units creating said interactive and adaptable spectrums creating the actual incidences and prevalences and propagations of causal relations of said pathognomonic adverse reactions, comprising:
interactive digital scrolls displaying list of interactive pharmaceutical product categories;
interactive digital scrolls displaying list of interactive digital scrolls pharmaceutical brand names and
interactive digital scrolls displaying list of interactive digital scrolls pharmaceutical generic names;
select buttons creating and adapting selections of said relational data in said plurality of interactive digital scrolls;
“x” buttons deleting thence adapting said selections;
submit buttons submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical brand names relating to pharmaceutical generic names in said relational databases;
said interactive digital scrolls-created relational data effecting said processor units getting relational data relating to
said interactive digital scrolls-created relational data in said relational databases;
said computer programs instructing processor units:
scanning for same pathognomonic adverse reactions in relational data relating to said brand names relating to a person's relational profile data and identification of abiotic intelligence-networked communication devices;
scanning for same pathognomonic adverse reactions in relational data relating to said generic names relating to a person's relational profile data and identification of abiotic intelligence-networked communication devices;
creating said interactive and adaptable spectrums associating with and relating to interactive highlighted and accentuated APS coordinates wherein same pathognomonic adverse reactions associating with and relating to interactive highlighted and accentuated APS coordinates in and among in figures, medical imagings and 2D & 3D holograms in and among viewing screens and touchscreens and ambiences displaying interactive 2D and 3D holograms;
same pathognomonic adverse reactions associating with and relating to interactive highlighted and accentuated APS coordinates in individual groupings of plurality of medications comprising Pharmaceutical Product Categories, Pharmaceutical brand names and Pharmaceutical generic names relating to people's relational profile data and identifications of said abiotic intelligence-networked communication devices;
Pharmaceutical Product Categories, Pharmaceutical brand names and Pharmaceutical generic names and medications-specific pathognomonic adverse reactions associating with and relating to interactive highlighted and accentuated APS coordinates, in individual polypharmaceuticals relating to individual persons and identifications of said abiotic intelligence-networked communication devices;
said people's relational profile data; and
said identifications of said abiotic intelligence-networked communication devices.

17. The method according to claim 12 whereby said processes of interactive and adaptable spectrums preventing and resolving pathognomonic adverse reactions comprising:
said interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates.

18. The method according to claim 12 whereby said processes of connected constellations of connected assortments of interactive and adaptable SIVSF-silhouetted SIUNDV comprising:
said interactive and adaptable spectrums associating with and relating to interactive highlighted and accentuated APS coordinates;
interactive highlighted and accentuated APS coordinates associating with said connected constellations of connected assortments of interactive and adaptable SIVSF-silhouetted SIUNDV comprising said interactive and adaptable spectrums, comprising:
said interactive and adaptable SIVSF comprising individual groupings of polypharmaceuticals wherein each medication comprising the following algorithm:
Pharmaceutical Product Category:
Pharmaceutical Generic Name;
Pharmaceutical Brand Name:
Actual Frequent Pathognomonic Adverse Reactions; adverse reaction of organ systems;
Actual Infrequent Pathognomonic Adverse Reactions; adverse reaction of organ systems;
Actual Rare Pathognomonic Adverse Reactions; adverse reaction of organ systems;
said interactive and adaptable silhouetted SIUNDV comprising the following algorithm:
Pharmaceutical Product Category*_m:
Pharmaceutical Generic Name*_n;
Pharmaceutical Brand Name*_n:
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
wherein said interactive and adaptable SIVSF silhouetting said interactive and adaptable silhouetted SIUNDV.

19. The method according to claim 12 whereby said processes of interactive and adaptable spectrums extrapolating the propagations and foretells of causal relations of pathognomonic adverse reactions in individual groupings of polypharmaceuticals associating with and relating to interactive highlighted and accentuated APS coordinates comprising:
said interactive and adaptable spectrums of pharmacologic basis of pathognomonic adverse reactions associating with and relating to individual groupings of polypharmaceuticals, wherein said interactive and adaptable spectrums associating with and relating to interactive highlighted and accentuated APS coordinates, comprising the following algorithm:
Pharmaceutical Product Category*_m;
Pharmaceutical Generic Name*_n;
Pharmaceutical Brand Name*_n;
Actual Frequent Pathognomonic Adverse Reaction*_p; APS coordinate*_q; adverse reaction of organ systems*;
Actual Infrequent Pathognomonic Adverse Reaction*_r; APS coordinate*_s; adverse reaction of organ systems*;
Actual Rare Pathognomonic Adverse Reaction*_t; APS coordinate*_u; adverse reaction of organ systems*;
interactive digital scrolls displaying list of interactive pharmaceutical product categories;
interactive digital scrolls displaying list of interactive digital scrolls pharmaceutical brand names and
interactive digital scrolls displaying list of interactive digital scrolls pharmaceutical generic names;
select buttons creating and adapting selections of said relational data among said plurality of interactive digital scrolls;
“x” buttons deleting thence adapting said selections;
submit buttons submitting said selections into said relational databases in and shared among said storage memories;
said interactive digital scrolls-created relational data comprising pharmaceutical product categories relating to pharmaceutical brand names in said relational databases;
said interactive digital scrolls-created relational data comprising pharmaceutical product categories relating to pharmaceutical generic names in said relational databases;
said interactive digital scrolls-created relational data effecting said processor units creating compilations of said incidences and prevalences of said actual, infrequent and rare pathognomonic adverse reactions associating with and relating to each and every pharmaceutical in individual grouping of polypharmaceuticals
compilations of said incidences and prevalences of propagations and foretells of causal relations in individual groupings of polypharmaceuticals in said relational databases;
commercial software including commercial statistical and actuarial software conferring statistical confidences of said extrapolations of the propagations and foretells of causal relations.
